(19) United States
(12) Patent Application Publication
(10) Pub. No.: US 2006/0084866 A1
(43) Pub. Date: Apr. 20, 2006

(54) Expanding imaging probe

(76) Inventors: Gadi Lewkonya, Neve-Mivtach (IL); Yuval Zur, Haifa (IL); Hanna Friedman, Givat-Zeev (IL); Aharon Blank, Kiryat-Ono (IL)

Correspondence Address: WOLF, BLOCK, SCHORR & SOLIS-COHEN LLP, 250 PARK AVENUE, NEW YORK, NY, 10177, US

(21) Appl. No.: 10/968,828

(22) Filed: Oct. 18, 2004

(51) Int. Cl. A61B 6/00

(52) U.S. Cl. 600/433

(57) ABSTRACT

An imaging probe for imaging inside a cavity surrounded by a wall, the probe comprising: a) a probe body having a contracted state and an expanded state; and b) at least two imaging sensors, mounted on the probe body and having fields of view in different directions; wherein, when the probe body is in the expanded state, the fields of view of the imaging sensors respectively comprise portions of the wall of the cavity on different sides of the cavity.

### RELATED APPLICATIONS

This application is related to a patent application titled “Magnetic Coil Configurations for MRI Probes,” attorney's docket number 334/03511, filed on even date, at the US Patent and Trademark Office, the disclosure of which is incorporated herein by reference.

### FIELD OF THE INVENTION

The field of the invention is imaging probes, especially medical imaging probes adapted for use in blood vessels and in other cavities.

### BACKGROUND OF THE INVENTION

MRI has advantages over other medical imaging methods in that it does not use ionizing radiation, and can distinguish better between different kinds of soft tissue than x-rays or ultrasound. A disadvantage of conventional MRI is that it requires a large, expensive magnet with limited mobility. It also has limited spatial resolution, with voxels typically about 1 mm on a side, due to limits on the RF field strength that can be used (to avoid overheating of body tissue and peripheral nerve stimulation), the distance from the RF coils to the region being imaged inside the body, and the limited time that most patients can tolerate staying inside the bore of a magnet without moving. Even if the patient does not move, parts of the body move internally, for example with the cardiac cycle, and temporal gating cannot compensate perfectly for this motion. These limitations prevent conventional MRI from being used to detect plaque in the wall of the arteries, for example, where a resolution of the order of 0.1 mm is required. Conventional MRI also has limited capability of measuring diffusion, and can measure diffusion rates of medical interest only by pushing the limits of conventional gradient coils.

In order to overcome these limitations of conventional medical MRI, MRI probes which are inserted into the body of a patient have been designed, for example, probes which go into the blood vessels, into the digestive track, or into other body cavities. In most cases, these probes have only RF receiving antennas, and conventional external magnets and RF coils are still used to apply the static magnetic field and to transmit the RF magnet fields that are used to excite the nuclei. See, for example, E. Atalar et al, “High Resolution Intravascular MRI/MRS using a Catheter Receiver Coil,” MRM 36(4), 596-605 (1996). This may improve the spatial resolution possible with MRI, but does not eliminate the need for large, expensive magnets. In other cases, for example U.S. Pat. No. 5,572,132 to Pulyer, MRI probes have been designed that are fully self-contained, with their own magnets (usually permanent magnets), RF transmitting and receiving coils (often the same coil), and even gradient coils, eliminating the need for large magnets and RF transmitting coils.

Westphal et al, in U.S. Pat. No. 5,959,454, describes a an MRI probe with an external imaging region on one side, for use outside the body to examine skin, for example. Crowley, in U.S. Pat. Nos. 5,304,930 and 5,517,118, describes MRI probes used outside the body, for imaging a part of the body, in which the magnetic field gradient is large, and the nuclei do get out of phase very quickly. Prado et al, in U.S. Pat. No. 6,489,767, describes a palm-sized MRI probe with a planar imaging region on one side.

Golan, in WO 01/42807, and Blank et al in WO 02/39132 A1, describe self-contained intravascular MRI probes which use thousands of spin echoes to obtain high signal to noise ratio in a high gradient magnetic field. Other types of medical imaging probes used inside the body are also known. For example, U.S. Pat. No. 6,059,731 to Seward is one of many publications describing a phased array of ultrasound transducers which is inserted into a blood vessel. Another example is Yock, P. G. and Linker, D. T., “Looking Below the Surface of Vascular Disease,” Circulation 81(5): 1715-1718 (May 1990).

There are various types of non-imaging sensors which have been used in conjunction with intravascular probes. U.S. Pat. No. 4,752,141, for example, describes a probe with a contact temperature sensor, to detect the elevated temperature of inflamed plaque in arteries.

U.S. Pat. No. 6,475,159 describes one or more infrared temperature sensors for detecting plaque in arteries, in which a transparent balloon, surrounding the sensors, expands to make contact with the wall, and the infrared sensors view one or more locations on the wall through the balloon.

In other cases, for example in U.S. Pat. Nos. 5,265,606 and 5,284,138, a sensor (in this case a blood gas sensor) must be kept away from the blood vessel wall in order to provide accurate measurements of oxygen or carbon dioxide concentration, or pH level.

There are intravascular probes, some of them with sensors of various types, which expand against the wall of the vessels in order to perform therapeutic functions.

U.S. Pat. Nos. 6,306,141 and 6,533,805, both to Jervis, describe stents made of superelastic NiTi, which are mechanically manipulated to expand inside arteries that are partially blocked by plaque. U.S. Pat. No. 5,197,978 to Hess, and 5,466,242 to Mori, also describe stents made of NiTi, but using the shape-memory temperature effect instead of the superelastic effect. U.S. Pat. No. 6,053,873, to Govari and Fenster describes a stent which expands inside an artery, with pressure and blood-flow sensors, the latter using non-imaging ultrasound transducers to make Doppler measurements.

U.S. Pat. No. 6,036,689, to Tu et al, describes a catheter with electrodes that expand against the walls of an artery, using either a mechanically expanding basket or a balloon, and use RF energy to ablate plaque. The electrodes have a temperature sensor to allow control of the ablation process.

U.S. Pat. No. 4,841,977, to Griffith et al, describes a catheter which performs balloon angioplasty, and has an ultrasound transducer array, surrounded by the balloon, which images the procedure in real time.

U.S. Pat. No. 6,542,781, to Koblish et al, describes helical or loop structures, made either of NiTi or other materials, which expand to press against the inside of a pulmonary vein and produce a circular lesion going all the way around, for example by RF heating, in order to treat atrial fibrillation. Temperature sensors are used to provide feedback for the heating. The helical and loop structures can also be used to push other diagnostic or therapeutic elements against the wall of a blood vessel.

U.S. Pat. No. 6,152,899 describes a catheter for shrinking veins, which has expandable arms, each with an electrode and a thermocouple. The electrodes heat the wall of the vein from the inside, using the thermocouples for feedback control of the temperature, and shrink the vein, with the expandable arms collapsing as the vein shrinks.

All of the foregoing patents, applications, and other publications are incorporated herein by reference.

### SUMMARY OF THE INVENTION

An aspect of some embodiments of the invention concerns an imaging probe in a blood vessel, or another cavity, with two or more imaging sensors looking in different directions. As used herein, an imaging sensor is a sensor which distinguishes sensing data from a plurality of different directions or locations, optionally arranged in the form of pixels or voxels. The probe expands, pushing the imaging sensors in different directions against the walls of the blood vessel or other cavity, where they each image a different part of the wall, with a different range of azimuthal angles. In one embodiment, the probe is an MRI probe in an artery, and the sensors, each a self-contained MRI device with a magnet and at least one RF antenna capable of RF transmitting and receiving, to provide data for producing images of plaque. Alternatively, one or more of the sensors have separate transmitting and receiving antennas.

Alternatively, other RF coupling elements are used for receiving and transmitting RF fields, instead of or in addition to antennas, for example Hall effect, magneto-optical, piezoelectric and magnetostrictive sensors and actuators, and micromachined mechanical structures resonant at RF frequencies. As used herein, “RF coupling element” refers to any such means for receiving or transmitting RF electric and magnetic fields, including an antenna, while “antenna” refers specifically to an element which directly couples to an electric field, or couples inductively to a magnetic field, for example a whip antenna or a loop antenna. It should be understood that, generally, whenever antennas are mentioned herein, other RF coupling elements are optionally used instead or in addition, in other embodiments of the invention.

Even if each sensor has a limited azimuthal field of view, as in the MRI probes described by Golan and by Blank et al, a broad azimuthal range of the wall can be imaged, in an embodiment of the invention, possibly even a full 360 degrees, by combining the data from the different sensors. There is optionally no need to rotate the probe, or to use an azimuthal phase encoding gradient.

The different imaging sensors need not all be located at the same longitudinal position along the blood vessel. Plaque in arteries tends to extend longitudinally over a much greater distance than its azimuthal and radial extent. Thus, if there are two or more sensors, looking at different azimuthal ranges of the wall, and located at different longitudinal positions but not too far apart longitudinally, then these sensors can be used to produce an image of plaque in the r-θ plane almost as if all the sensors were located at the same longitudinal position.

For example, in an embodiment of the invention, there is a sub-probe comprising two sensors at one longitudinal position, looking at azimuthal ranges that are centered 180 degrees apart from each other (in the +x and −x directions), and a second sub-probe comprising two more sensors, at a different longitudinal position, which look at azimuthal ranges that are 180 degrees from each other and 90 degrees from the directions of the first two sensors (in the +y and −y directions). The four sensors together cover a large fraction or even all of 360 degrees around the artery wall. For example, if each sensor has an azimuthal field of view that is between 45 and 60 degrees wide (measured from the center of the artery, not from the center of the sensor) then together the four sensors cover between 180 and 240 degrees around the artery wall.

The present invention is not limited to MRI probes, but concerns other types of imaging probes, for example ultrasound probes, which are pressed against different sides of a blood vessel or other cavity.

Another aspect of some embodiments of the invention concerns a series of sub-probes, each comprising a pair of sensors, such as the sub-probes described in the previous paragraph. Each sub-probe is located at a different longitudinal position on a probe. Within each sub-probe, the pair of sensors is joined to an expansion mechanism which makes the sensors move away from each other to image opposite sides of a blood vessel or another lumen. A single control element, such as a control cable, incorporated into a catheter, is manipulated to make the different sub-probes expand simultaneously.

Optionally, each sensor is an imaging sensor, obtaining data from more than one voxel. Alternatively, each sensor does not obtain data from more than one voxel, and the data from the different sensors is not used to construct an image, but is used, for example, to find an average value, or a maximum or minimum value, or a distribution of values, of some parameter in the vicinity of the probe. Alternatively, even if each sensor only obtains data from one voxel,an image is constructed from the probe as a whole by combining data from the different sensors. For example, each sensor is a non-imaging NMR sensor, obtaining data from a single voxel, or each sensor is a thermal sensor, measuring the temperature of the wall of the blood vessel at its location.

Optionally, the control cable is coupled to the expansion mechanism for each sub-probe by means of an adaptive mechanism, such as individual springs, which allows each sub-probe to adapt to the inner diameter of the blood vessel at that longitudinal location, when it expands. If there were no adaptive mechanism and the lumen varies in diameter along its length, then all the sub-probes would only open as far as the narrowest part of the lumen, and the other sub-probes would not reach the wall. Or, possibly, the sub-probes in the narrower parts of the lumen would push against the wall so hard that they would distort the blood vessel (possibly breaking the plaque), enough so that these sub-probes could expand as much as the sub-probes in the wider parts of the lumen. With the adaptive mechanism, the sensors in different sub-probes press firmly enough against the wall to take reliable data, without pressing hard enough to distort the wall.

Another aspect of some embodiments of the invention concerns an intravascular probe, such as those described above with one or more pairs of sensors, which expands so that it touches two sides of a blood vessel. The outer surface of the probe comprises a sheath, to keep blood from coming into contact with components of the probe that may not be bio-compatible, and there is no passage by which blood can flow through the center of the probe. The probe is not in contact with the vessel wall over the entire circumference of the vessel, but only in two contact regions on opposite sides of the vessel. Between these two contact regions, there are contact-free regions through which blood can flow around the probe.

If the probe includes another pair of sensors further along longitudinally, which expands at right angles to the first pair of sensors, then the free regions and contact regions will be at different azimuthal angles at different longitudinal positions, but the free regions still form a continuous volume along which blood can flow.

An aspect of some embodiments of the invention concerns an MRI probe comprising a magnet or set of magnets in the shape of a cylinder (not necessarily a right circular cylinder), with slots carved out of the magnet for RF antennas, for example coils. The RF coils fit entirely into the slots, so that the magnet together with the coils fits into the smallest convex volume that contains the magnet, viz. the cylindrical shape of the magnet before the slots were carved out. This allows the probe to be inserted easily into a blood vessel, and also allows the surface of the magnet, with its high field, to be pressed against the wall of an artery being imaged, except perhaps for a thin sheath that covers the magnet, for example if the magnet is not bio-compatible. For probes which have high magnetic field gradients, it is potentially advantageous for the surface of the magnet to be close to the wall, in order to make the magnetic field as high as possible in the region of the wall that is being imaged, and in order to make the imaging region extent as far as possible into the wall.

The slots and coils optionally do not extend over the entire length of the magnet, but each slot extends over less than the length of the magnet. This may allow the static magnetic field to be higher in the imaging region than if the slots and coils extended over the whole length of the magnet.

There is thus provided, in accordance with an exemplary embodiment of the invention, an imaging probe for imaging inside a cavity surrounded by a wall, the probe comprising:
a) a probe body having a contracted state and an expanded state; and
b) at least two imaging sensors, mounted on the probe body and having fields of view in different directions;
   wherein, when the probe body is in the expanded state, the fields of view of the imaging sensors respectively comprise portions of the wall of the cavity on different sides of the cavity.

Optionally, when the probe body is in the expanded state, the at least two imaging sensors are displaced from each other toward the wall, from their position when the probe body is in the contracted state.

Optionally, the probe is adapted for inserting into a blood vessel and using the blood vessel as the cavity.

Optionally, the probe includes a biocompatible sheath which covers the probe.

Optionally, the sheath keeps blood from coming into contact with and flowing through the probe body.

In an embodiment of the invention, when the probe body is in the expanded state, the probe touches the wall of the blood vessel in two contact regions on two opposite sides of the vessel, while leaving at least one free region, where the probe is not in contact with the wall, between the contact regions, thereby allowing blood to flow around the probe through the at least one free region.

Optionally, the probe has a diameter between 1 and 2 mm in its contracted state.

Optionally, the probe has a diameter between 2 mm and 6 mm in its expanded state.

Optionally, the diameter of the probe in its expanded state is at least 1.5 times the diameter of the probe in its contracted state.

In an embodiment of the invention, the imaging sensors are MRI sensors, each sensor comprising:
a) at least one static magnetic field source which creates a static magnetic field in the field of view of said sensor; and
b) at least one RF coupling element, wherein at least one of the at least one RF coupling elements is capable of creating a time-varying magnetic field which is capable of exciting nuclei in the field of view of said sensor, and at least one of the at least one RF coupling elements is capable of receiving NMR signals from said excited nuclei and generating NMR electrical signals therefrom.

Optionally, at least one of the at least one RF coupling elements comprises an antenna.

Optionally, the antenna comprises a coil.

Alternatively or additionally, at least one of the at least one RF coupling elements uses the Hall effect.

Alternatively or additionally, at least one of the at least one RF coupling elements uses the magneto-optical effect.

In an embodiment of the invention, for at least one of the MRI sensors:
a) the at least one static magnetic field source comprises at least one permanent magnet;
b) the convex magnet volume, defined as a smallest convex volume which includes all of the at least one magnet, is cylindrical;
c) the at least one magnet substantially reaches all of the radial surface of the convex magnet volume, except for at least one slot, each slot being less than the length of the convex magnet volume; and
d) one of the at least one RF coupling elements is located in one of the at least one slots, substantially entirely within the convex magnet volume.

Optionally, for each of the MRI sensors, the at least one static magnetic field source comprises a permanent magnet.

Optionally, the magnets of the two MRI sensors repel each other.

Optionally, the magnets of the two MRI sensors are both magnetized in directions that are more than 45 degrees away from an axis along which the sensors move apart from each other when the probe body expands, and the magnets are magnetized in directions less than 90 degrees away from each other.

Alternatively, the magnets of the two MRI sensors are both magnetized in directions that are less than 45 degrees away from an axis along which the sensors move apart from each other when the probe body expands, and the magnets are magnetized in directions more than 90 degrees away from each other.

Optionally, the time-varying magnetic field created by the at least one RF coupling element of each MRI sensor is oriented at an angle between 45 and 135 degrees from the direction of the static magnetic field created by the static magnetic field source of said MRI sensor, at at least one location in the field of view of said MRI sensor.

Alternatively or additionally, the imaging sensors are ultrasound imaging sensors.

Optionally, the at least two imaging sensors comprise exactly two imaging sensors.

Alternatively, the at least two imaging sensors comprise at least three imaging sensors.

In an embodiment of the invention, the probe body comprises a plurality of expansion mechanisms, each expansion mechanism attached to at least two but not all of the imaging sensors, such that when each expansion mechanism causes the imaging sensors to which it is attached to move apart from each other, the probe body expands.

Optionally, the imaging sensors are arranged in a circle, and one of the expansion mechanisms is located between, and attached to, each pair of adjacent imaging sensors in the circle.

Optionally, at least one expansion mechanism comprises a pair of leaf springs.

Optionally, at least one of the expansion mechanisms comprises shape memory alloy.

Alternatively, the probe body comprises a single centrally located expansion mechanism which is attached to all the sensors, and causes the sensors to move apart from each other, expanding the probe.

Optionally, the expansion mechanism comprises a basket comprising a plurality of arms, each arm attached to exactly one sensor and each sensor attached to exactly one arm.

Optionally, the expansion mechanism comprises shape memory alloy.

Optionally, raising the temperature of the shape memory alloy above its transition temperature causes said expansion mechanism to expand.

Alternatively or additionally, said expansion mechanism operates using a superelastic effect of the shape memory alloy.

In an embodiment of the invention, the probe body comprises an expansion mechanism which causes the two sensors to move apart from each other, expanding the probe.

Optionally, the expansion mechanism comprises a pair of leaf springs joined at both their ends and free in their middle portions, and each sensor is attached to the middle portion of a different one of the leaf springs, and not attached to the other leaf spring.

There is further provided, in accordance with an exemplary embodiment of the invention, an imaging system comprising an imaging probe as described, and a catheter adapted for inserting the imaging probe into the cavity.

Optionally, the catheter comprises a control cable, and manipulating the control cable causes the probe body to expand and contract.

Optionally, the imaging system comprising a plurality of sub-probes, each sub-probe being an imaging probe as described, and a catheter adapted for inserting the sub-probes into the cavity.

Optionally, the catheter comprises a control cable, and manipulating the control cable causes the probe body of at least two of the sub-probes to expand and contract.

Optionally, the control cable is coupled to the sub-probes in a manner such that manipulating the control cable causes the probe bodies of a plurality of the sub-probes to expand simultaneously, and to contract simultaneously.

Optionally, for each sub-probe in said plurality, one or both of said sub-probe and its coupling to the control cable is sufficiently flexible so that, when the control cable is manipulated, each sub-probe in said plurality expands to an extent that depends on the distance to the walls of the cavity, at the location of that sub-probe.

Optionally, for each sub-probe in said plurality, one or both of said sub-probe and its coupling to the control cable is sufficiently flexible so that, if the cavity is any artery the inner diameter of which varies between 2 mm and 4 mm at the locations of the sub-probes in said plurality, then all of the sub-probes in said plurality will touch the inner walls of the artery when the control cable is manipulated to cause said plurality of sub-probes to expand, without exerting a pressure of more than 1 atmosphere on the wall of the artery.

There is further provided, in accordance with an exemplary embodiment of the invention, a method of producing images of the walls of a cavity, comprising:
a) introducing an imaging probe comprising a plurality of imaging sensors into the cavity;
b) causing the imaging probe to expand, causing the imaging sensors to move away from each other toward the walls;
c) generating imaging data by each imaging sensor in a different field of view, adjacent to that imaging sensor, of the walls of the cavity; and
d) reconstructing an image of the walls of the cavity from the imaging data.

Optionally, introducing an imaging probe into the cavity comprises introducing the imaging probe into a lumen.

Optionally, introducing the imaging probe into a lumen comprises introducing the imaging probe into a blood vessel.

Optionally, causing the imaging probe to expand comprises causing the imaging probe to touch the wall of the blood vessels at a contact region, and leaving a free region where the imaging probe does not touch the blood vessel wall, allowing blood to flow around the imaging probe.

Optionally, causing the imaging probe to expand comprises causing each of a plurality of sub-probes to expand by different amounts, depending on the inner diameter of the lumen at the location of each of said sub-probes.

Optionally, introducing the imaging probe into the cavity comprises using a catheter.

Optionally, causing the imaging probe to expand comprises manipulating the catheter.

Optionally, generating imaging data comprises transmitting electrical power to the imaging probe through the catheter.

Optionally, generating imaging data comprises receiving imaging data from the imaging probe through the catheter.

Optionally, reconstructing an image comprises analyzing data by a data analyzer, and including transmitting the imaging data from the imaging sensors to the data analyzer, wherein the data from at least two of the sensors is transmitted on a same cable.

Optionally, the sensing data from said two sensors is transmitted at different times.

Alternatively or additionally, the sensing data from said two sensors is transmitted in different frequency bands.

In an embodiment of the invention, the method includes digitally encoding the data from said two sensors into different digital channels before transmitting it, and decoding the data from said two sensors after transmitting it, before analyzing it.

There is further provided, in accordance with an exemplary embodiment of the invention, a probe adapted for inserting into a lumen, comprising a plurality of sub-probes, each having a contracted state, and a plurality of expanded states in each of which the sub-probe expands to a different extent, wherein each sub-probe is adapted to expand to an extent that depends on an inner diameter of the lumen, at the location of that sub-probe.

Optionally, the probe also includes a control cable, coupled to each of the sub-probes, which control cable, when it is manipulated, causes each of the sub-probes to expand, wherein for each sub-probe, one or both of said sub-probe and its coupling to the control cable are sufficiently flexible so that, when the control cable is manipulated, each sub-probe expands to the extent that depends on the inner diameter of the lumen at the location of that sub-probe.

Optionally, each sub-probe has a distal end and a proximal end, and manipulating the control cable shortens the distance between the distal end and proximal end of each sub-probe, thereby causing a middle portion of each sub-probe between the distal and proximal ends to bow outward, expanding that sub-probe.

In an embodiment of the invention, manipulating the control cable to expand the sub-probes allows the center of each sub-probe to remain in substantially a fixed position along the blood vessel.

Optionally, the control cable comprises:
a) a first portion coupled to the distal end of each sub-probe; and
b) a second portion coupled to the proximal end of each sub-probe; whereby manipulating the control cable to expand the sub-probes comprises pulling on the first portion relative to the second portion.

Optionally, every expanded sub-probe returns to its contracted state when no pulling force is applied to the first portion relative to the second portion.

Optionally, a force between 0.5 and 2 newtons pulling on the first portion relative to the second portion is necessary and sufficient to fully expand all the sub-probes when there is no external force on the sub-probes resisting their expansion.

Optionally, a force between 0.5 and 2 newtons pulling on the first portion relative to the second portion is necessary and sufficient to expand all the sub-probes by a factor of 2 in diameter, when there is no external force on the sub-probes resisting their expansion.

Optionally, the second portion comprises a cable sheath surrounding the first portion which comprises an inner cable.

Optionally, the cable sheath includes a hole adjacent to the distal portion of each sub-probe, through which hole the inner cable is coupled to said distal portion.

Optionally, for at least one sub-probe, the first portion is coupled to the distal portion of that sub-probe through a distal adaptive spring, whereby, when the cable is manipulated, that sub-probe expands to an extent that depends on the inner diameter of the lumen, at the location of that sub-probe.

Alternatively or additionally, for at least one sub-probe, the second portion is coupled to the proximal portion of that sub-probe through a proximal adaptive spring, whereby, when the cable is manipulated, that sub-probe expands to an extent that depends on the inner diameter of the lumen, at the location of that sub-probe.

Optionally, at least one sub-probe comprises a pair of leaf springs.

Alternatively or additionally, at least one sub-probe comprises a basket structure.

Optionally, at least one sub-probe comprises shape memory alloy.

Optionally, the shape memory alloy is superelastic.

Optionally, at least one of the sub-probes has a diameter between 1 and 1.5 mm in its contracted state.

Optionally, said sub-probe has a diameter between 1.7 mm and 6 mm in its maximally expanded state.

Optionally, at least one of the sub-probes has a diameter between 1.7 mm and 6 mm in its maximally expanded state.

Optionally, the probe includes a plurality of sensors attached to at least one of the sub-probes, which sensors each generate sensing data from a different portion of the wall of the lumen, when said sub-probe is expanded sufficiently so that said sensors are adjacent to the wall.

Optionally, at least one of the sensors is a non-imaging NMR sensor.

Alternatively or additionally, at least one of the sensors is a thermal sensor.

Optionally, the plurality of sensors comprises sensors attached to at least two of the sub-probes.

Optionally, at least two of the sub-probes each have at least two of the sensors attached to them, and each of said sub-probes is adapted to expand to an extent such that each of the two sensors is adjacent to a different portion of the wall.

There is further provided, in accordance with an exemplary embodiment of the invention, a method of obtaining sensing data from an extended region of the wall of a lumen, comprising:
a) inserting a probe as described into the lumen;
b) manipulating the control cable so that at least two of the sensors on each of at least two of the sub-probes are adjacent to the wall of the lumen; and
c) generating sensing data by said sensors from the different portions of the wall of the lumen.

Optionally, the method includes transmitting the sensing data from the sensors to a data analyzer, wherein the sensing data from at least two of the sensors is transmitted on a same cable.

Optionally, at least one of the sensors is an imaging sensor.

Optionally, the imaging sensor is an MRI sensor.

There is further provided, in accordance with an exemplary embodiment of the invention, an imaging system for imaging the walls of a lumen, comprising:
a) a probe as described;
b) a power supply capable of supplying power at least at an RF frequency;
c) a power channel which conveys electrical power from the power supply to the imaging sensors;
d) a receiving channel; and
e) a controller which controls one or more of the timing, amplitude, frequency and phase of the electrical power, and which receives imaging data from the imaging sensors through the receiving channel.

Optionally, the imaging system includes a catheter which holds together the control cable, the transmitting channel, and the receiving channel.

Optionally, the catheter is adapted for inserting the probe into the lumen.

There is further provided, in accordance with an exemplary embodiment of the invention, a probe adapted for inserting into a blood vessel, comprising:
a) a first sub-probe body having a contracted state and an expanded state; and
b) a sheath which covers the first sub-probe body and keeps blood from coming into contact with and flowing through the first sub-probe body;
   wherein when the first sub-probe body is in the expanded state, the probe touches the wall of the blood vessel in a first contact region and a second contact region on two opposite sides of the blood vessel, while leaving at least a first free region, where the probe is not in contact with the wall, between the contact regions, thereby allowing blood to flow around the probe at least through the first free region.

Optionally, the sheath comprises silicone.

Alternatively or additionally, the sheath comprises polyurethane.

Alternatively or additionally, the sheath comprises SCBS.

Alternatively or additionally, the sheath comprises a composite material.

Optionally, the sheath is between 10 and 100 micrometers thick.

Optionally, when the probe touches the wall of the blood vessel in the first and second contact regions, it leaves a second free region on an opposite side of the blood vessel from the first free region, thereby allowing blood to flow around two sides of the probe.

Optionally, the probe includes a second sub-probe body, having a contracted state and an expanded state, located at a different longitudinal location from the first sub-probe body, wherein the sheath also covers the second sub-probe body and keeps blood from coming into contact with and flowing through the second sub-probe body, and wherein, when the second sub-probe is in its expanded state, the probe comes into contact with the wall in a third contact region and a fourth contact region, on opposite sides of the blood vessel, leaving a third free region between the third and fourth contact regions, thereby allowing blood to flow around the probe at the longitudinal location of the second sub-probe body.

Optionally, when the probe touches the wall of the blood vessel in the third and fourth contact regions, it leaves a fourth free region on an opposite side of the blood vessel from the third free region, thereby allowing blood to flow around two sides of the probe at the longitudinal location of the second sub-probe body.

Optionally, the direction from the first contact region to the second contact region, and the direction from the third contact region to the fourth contact region, excluding any longitudinal components, differ from each other by more than 10 degrees and less than 170 degrees.

Optionally, the free regions connect to form a continuous passage within which blood can flow past the entire length of the probe.

Optionally, the surface of the sheath does not have pockets where blood stagnates.

In an embodiment of the invention, the probe includes a second sub-probe body, located at a different longitudinal location from the first sub-probe body when the probe is inserted in the blood vessel, the second sub-probe body having a contracted state and a plurality of expanded states, and the first sub-probe body has a plurality of expanded states, the first and second contact regions are at the longitudinal location of the first sub-probe body, and the two sub-probe bodies are adapted so that when the first sub-probe body is in the expanded state in which the probe touches the wall of the blood vessel in the first and second contact regions, then the second sub-probe body is in an expanded state in which the probe touches the wall in a third contact region and a fourth contact region, on opposite sides of the blood vessel, at the longitudinal location of the second sub-probe body.

Optionally, the probe also includes two imaging sensors mounted on the sub-probe body and having fields of view in different directions, and, when the sub-probe body is in the expanded state, the fields of view of the imaging sensors respectively comprise portions of the wall on different sides of the blood vessel.

There is further provided, in accordance with an exemplary embodiment of the invention, an imaging system comprising a probe according to an embodiment of the invention, and a catheter adapted for inserting the probe into the blood vessel.

There is further provided, in accordance with an exemplary embodiment of the invention, an imaging system for imaging the walls of a blood vessel, comprising:
a) a probe according to an embodiment of the invention;
b) a power supply capable of supplying power at least at an RF frequency;
c) a power channel which conveys electrical power from the power supply to the imaging sensors;
d) a receiving channel; and
e) a controller which controls one or more of the timing, amplitude, frequency and phase of the electrical power, and which receives imaging data from the imaging sensors through the receiving channel.

There is further provided, in accordance with an exemplary embodiment of the invention, a method of obtaining sensing data from an extended region of the wall of a blood vessel, the method comprising:
a) inserting a probe according to an embodiment of the invention into the blood vessel;
b) manipulating the control cable so that at least two of the sensors on each of at least two of the sub-probes are adjacent to the wall of the blood vessel; and
c) generating sensing data by said sensors from the different portions of the wall of the blood vessel.

There is further provided, in accordance with an exemplary embodiment of the invention, a method of producing images of the wall of a blood vessel, the method comprising:
a) introducing a probe according to an embodiment of the invention into the blood vessel;
b) expanding the probe into the expanded state;
c) generating imaging data by each imaging sensor in its field of view; and
d) reconstructing an image of the wall of the blood vessel from the imaging data.

There is further provided, in accordance with an exemplary embodiment of the invention, a magnetic resonance sensor comprising:
a) at least one permanent magnet which creates a static magnetic field in an excitation region; and
b) at least one RF coupling element capable of creating a time-varying magnetic field which is capable of exciting nuclei in the excitation region, and capable of receiving

NMR signals from said excited nuclei and generating NMR electrical signals therefrom; wherein a smallest convex volume which includes all of the at least one magnet is substantially cylindrical, the at least one magnet substantially reaches all of the radial surface of the convex volume, except for at least one slot, each slot being less than the length of the convex volume, and one of the at least one RF coupling elements is located in one of the at least one slots, substantially entirely within the convex volume.

Optionally, at least one of the at least one RF coupling elements comprises an antenna.

Optionally, the at least one magnet comprises a sintered material whose skin depth, at the proton nuclear magnetic resonance frequency at the greatest field at the surface of the magnet, is at least twice the largest dimension of the magnet perpendicular to the cylindrical axis of the convex magnet volume.

Optionally, the at least one magnets substantially comprise only a single magnet, uniformly magnetized in a single direction.

Optionally, at least one of the at least one slots with at least one RF coupling element in it runs substantially perpendicular to the cylindrical axis of the convex magnet volume.

Optionally, the at least one RF coupling element in said slot comprises a coil.

Optionally, the time-varying magnetic field at the center of the coil is oriented substantially perpendicular to the direction of the slot and to the cylindrical axis.

Optionally, the magnet is magnetized substantially parallel to the direction of the slot, adjacent to the slot.

Optionally, the slot is less than half the length of the convex magnet volume.

There is further provided, in accordance with an exemplary embodiment of the invention, an imaging probe for imaging inside a cavity surrounded by a wall, the probe comprising:
a) a probe body having a contracted state and an expanded state; and
b) at least two magnetic resonance sensors according to an embodiment of the invention, adapted for MRI, mounted on the probe body and having fields of view in different directions; wherein, when the probe body is in the expanded state, the fields of view of the magnetic resonance sensors respectively comprise portions of the wall of the cavity on different sides of the cavity.

There is further provided, in accordance with an exemplary embodiment of the invention, an NMR system comprising:
a) an NMR probe comprising at least one magnetic resonance sensor according to an embodiment of the invention;
b) a power supply capable of supplying power at least at an RF frequency;
c) a transmitting channel which transmits electrical power from the power supply to at least one of the at least one RF coupling elements in the sensor, which RF coupling element excites nuclei in the excitation region;
d) a receiving channel; and
e) a controller which controls one or more of the timing, amplitude, frequency and phase of the electrical power, and which receives NMR data in the form of NMR electrical signals from at least one of the at least one RF coupling elements in the sensor, through the receiving channel.

Optionally, the NMR probe is adapted for use inside the body.

Optionally, the NMR probe is adapted for use as an intravascular NMR probe.

In an embodiment of the invention, the NMR system is adapted for imaging a a wall surrounding a cavity, the NMR data comprises imaging data, the NMR probe has a contracted state and an expanded state, the at least one magnetic resonance sensor comprises at least two magnetic resonance sensors, adapted for MRI, mounted on the NMR probe and having fields of view in different directions, and when the NMR probe is in the expanded state inside the cavity, the fields of view of the magnetic resonance sensors respectively comprise portions of the wall of the cavity on different sides of the cavity.

Optionally, for at least one slot, a same RF coupling element both excites nuclei in the excitation region, and receives NMR signals from said excited nuclei and generates NMR electrical signals therefrom.

Alternatively or additionally, for at least one slot, a first RF coupling element excites nuclei in the excitation region, and a second RF coupling element receives NMR signals from said excited nuclei and generates NMR electrical signals therefrom.

Optionally, the at least one slots comprise a plurality of slots, each with at least one RF coupling element, all of said plurality of slots running substantially in a same direction perpendicular to the cylindrical axis, and spaced apart in the direction of the cylindrical axis.

Optionally, the at least one RF coupling element in each of said plurality of slots comprises a coil.

Optionally, the time-varying magnetic field at the center of the coil in each of said plurality of slots is oriented substantially perpendicular to the direction of the slot and to the cylindrical axis.

Optionally, the magnet is magnetized substantially parallel to the direction of the slot, adjacent to the slot, for each of said plurality of slots.

Optionally, each of said plurality of slots is less than half the length of the convex magnet volume, in a direction parallel to the cylindrical axis.

Optionally, for at least two of the slots, the NMR electrical signals produced by the RF coupling elements in those slots are lumped together in the receiving channel.

Alternatively or additionally, for at least two of the slots, the NMR electrical signals produced by at least one RF coupling element in a first one of the two slots, and the NMR electrical signals produced by at least one RF coupling element in a second one of the two slots, are sent through the receiving channel in a manner that allows the controller to distinguish the two sets of signals.

Optionally, the controller uses the two sets of signals to reconstruct an image comprising separate pixels adjacent to the first slot and the second slot.

There is further provided, in accordance with an exemplary embodiment of the invention, a method of analyzing NMR signals from a viewing region which is extended in a longitudinal direction, comprising:
a) bringing a sensor as described into a position such that the excitation region of the sensor corresponds to the viewing region, and the cylindrical axis of the sensor is oriented in the longitudinal direction;
b) exciting nuclei in the excitation region using time-varying magnetic fields created by at least one of the at least one RF coupling elements in each of the plurality of slots;
c) receiving NMR signals from a portion of the excitation region adjacent to each of the plurality of slots, using at least one of the at least one RF coupling elements in said slot, and creating NMR electrical signals from said NMR signals;
d) selecting which slots to lump together and which slots to treat separately, according to a desired trade-off between signal to noise ratio and longitudinal resolution; and
e) analyzing the NMR electrical signals from the plurality of slots according to the selection.

Optionally, analyzing the NMR data comprises reconstructing an image of the viewing region with a plurality of pixels in the longitudinal direction.

Alternatively or additionally, analyzing the NMR data comprises obtaining an NMR spectrum of the viewing region.

Optionally, exciting nuclei in the excitation region comprises creating the time-varying magnetic fields at different times by the RF coupling elements that are in slots that are selected to be treated separately.

Alternatively or additionally, exciting nuclei in the excitation region comprises creating the time-varying magnetic fields in different frequency bands by the RF coupling elements that are in slots that are selected to be treated separately.

Optionally, the NMR electrical signals from RF coupling elements in slots that are selected to be treated separately are obtained from said RF coupling elements using separate cables.

Alternatively or additionally, the NMR electrical signals created by the RF coupling elements in slots that are selected to be treated separately are transmitted at different times.

Alternatively or additionally, the NMR electrical signals created by the RF coupling elements in slots that are selected to be treated separately are transmitted at different frequencies.

### BRIEF DESCRIPTION OF THE DRAWINGS

Exemplary, non-limiting, embodiments of the invention are described in the following sections with reference to the drawings. The drawings are generally not to scale and the same or similar reference numbers are used for the same or related features on different drawings.

FIGS. 1A, 1B and 1C are an ordered sequence of perspective views, showing an intravascular probe with expanding pairs of imaging sensors, according to an exemplary embodiment of the invention;

FIG. 2 is a schematic side cross-sectional view showing the details of part of the probe shown in FIGS. 1A, 1B and 1C;

FIGS. 3 is a perspective side view of the probe shown in FIG. 1C with a sheath covering it; and

FIG. 4 is a perspective view of an MRI imaging sensor according to an exemplary embodiment of the invention, which is usable, for example, in the embodiment shown in FIGS. 1A-1C;

FIG. 5 is a schematic side cross-sectional view of an MRI imaging sensor, according to an exemplary embodiment of the invention different from that in FIG. 4;

FIG. 6 is a cross-sectional view, in a plane perpendicular to the longitudinal axis, of a sub-probe comprising a pair of MRI sensors, showing the direction of magnetization of the magnets, for example for MRI sensors of the kind shown in FIG. 4 used in the imaging probes shown in FIGS. 1A-1C;

FIG. 7 is a cross-sectional view, in a plane perpendicular to the longitudinal axis, of a sub-probe comprising a pair of MRI sensors, according to an exemplary embodiment of the invention;

FIGS. 8, 9, and 10 are cross-sectional views, in a plane perpendicular to the longitudinal axis, of sub-probes comprising four MRI sensors, according to various exemplary embodiments of the invention;

FIG. 11 is a cross-sectional schematic view, in a plane perpendicular to the longitudinal axis, illustrating a possible mechanical instability in the embodiments shown in FIGS. 9, 10 and 11;

FIGS. 12, 13 and 14 are cross-sectional views, in a plane perpendicular to the longitudinal axis, of a sub-probe comprising a pair of MRI sensors, according to various exemplary embodiments of the invention; and

FIGS. 15 and 16 are cross-sectional views, in plane perpendicular to the longitudinal axis, of a sub-probe comprising three MRI sensors, according to various exemplary embodiments of the invention.

### DETAILED DESCRIPTION OF EXEMPLARY EMBODIMENTS

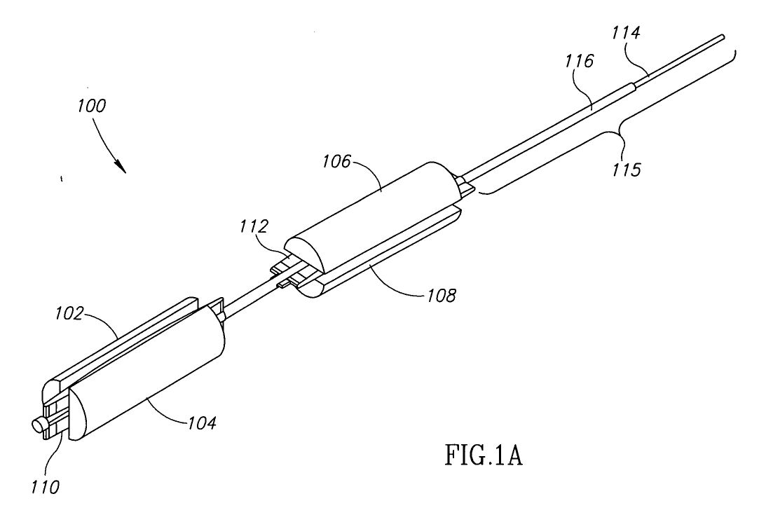

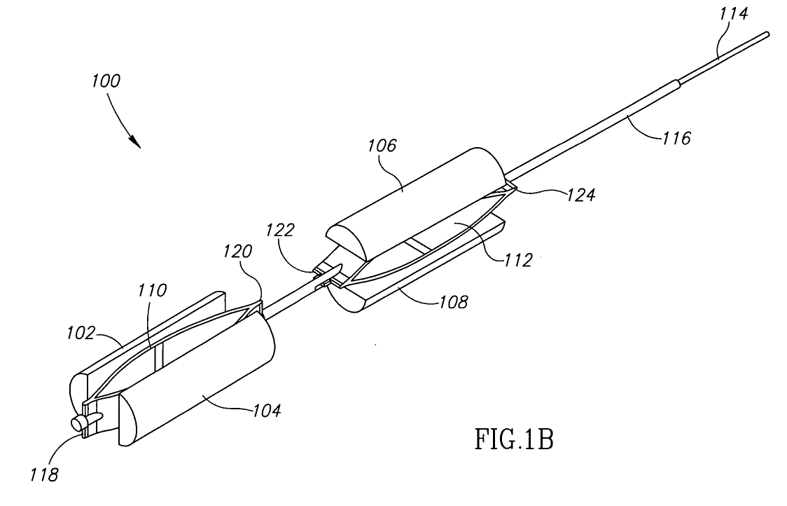

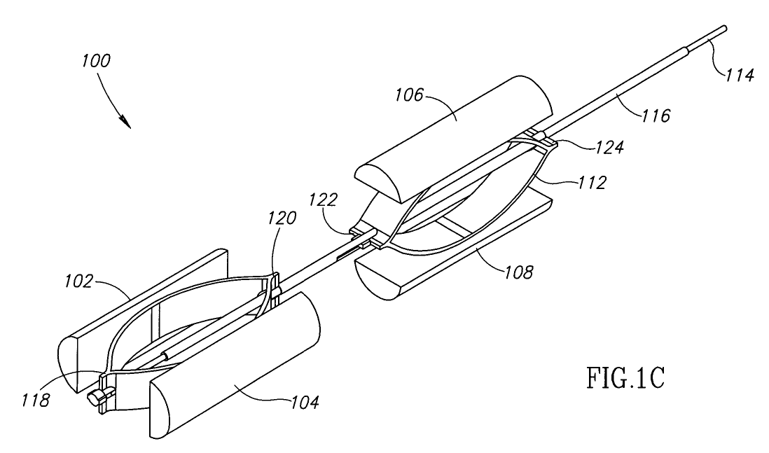

FIGS. 1A-1C show an intravascular probe 100, with four imaging sensors 102, 104, 106, and 108. Optionally, each sensor is an MRI probe, for example a self-contained MRI probe with its own magnet and one or more antennas (or other means) for transmitting and receiving RF power. Alternatively, other kinds of imaging sensors are used, for example MRI probes with only RF receiving antennas, or ultrasound probes. The imaging sensors are shown grouped in pairs. Sensors 102 and 104 are attached to a pair of leaf springs 110, comprising one sub-probe, and sensors 106 and 108 are attached to another pair of leaf springs 112, comprising a second sub-probe. A catheter 115 comprises an inner wire 114 and a sheath 116 surrounding the inner wire, which are coupled to the leaf springs.

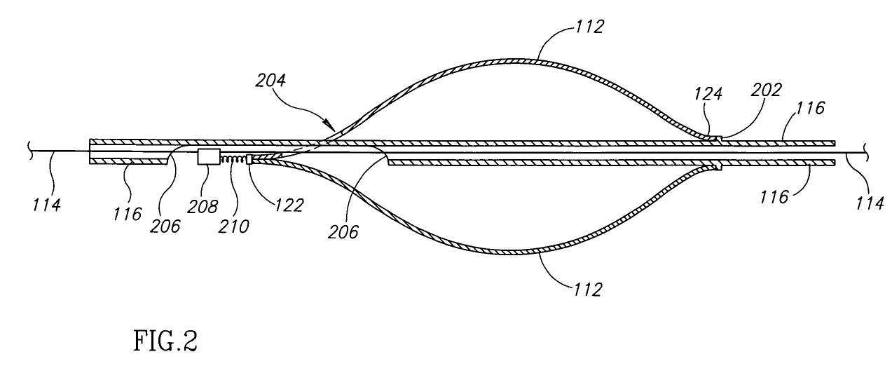

As shown most clearly in FIG. 2, when inner wire 114 is pulled back relative to sheath 116, the pair of leaf springs 110 expands, pushing sensors 102 and 104 away from each other, and the pair of leaf springs 112 expands, pushing sensors 106 and 108 away from each other. FIGS. 1B and 1C show the pairs of leaf springs successively more and more expanded. The pairs of leaf springs expand until the imaging sensors push against the walls of the blood vessel (not shown in the drawings) into which the probe is inserted. The two pairs of sensors shown in FIGS. 1A-1C expand in two perpendicular directions, so that the four sensors touch the wall of the blood vessel at four different azimuthal angles, 90 degrees apart. This allows the imaging sensor to collect imaging data covering much or all of a full 360 degrees in azimuth, depending on the azimuthal field of view of each probe.

Although the field of view of sensors 102 and 104 is at a different longitudinal location than the field of view of sensors 106 and 108, this may not matter very much, if the sensors are used to image plaque, and if plaque tends to extend longitudinally over a distance greater than the distance between the two pairs of sensors.

Data from the four sensors is optionally collected simultaneously. Alternatively, data is collected simultaneously from only some of the sensors, or data is collected serially, one sensor at a time. Data collected simultaneously from two or more sensors is optionally transmitted on a single cable (not shown) going through catheter 115, for example an electrical cable or an optical fiber, using different frequency bands for different sensors, or using any method known in the art of multiplexing to transmit more than one channel on a single cable, or the data is transmitted by one or more RF channels. However, whether the data is collected simultaneously or serially, it is not necessary to rotate the probe each time a different azimuthal field of view is imaged.

Optionally, instead of two pairs of sensors as shown in FIGS. 1A-1C, there is only one pair of sensors, or more than two pairs of sensors. If there are more than two pairs of sensors, then optionally each pair is oriented to expand in a different direction. For example there are three pairs of sensors, expanding in three different directions 120 degrees apart, and each sensor has a 60 degree field of view in azimuthal angle, so that all six sensors cover a full 360 degrees. Alternatively, there are two or more pairs of sensors, and two or more of the pairs are oriented to expand in a same direction, but are far enough apart longitudinally that both pairs will not, in general, see the same azimuthal and radial distribution of plaque. With this arrangement, it is possible to obtain imaging data at two or more different longitudinal positions, without the need to move the probe longitudinally between measurements.

Optionally, instead of or in addition to sub-probes comprising pairs of sensors, the sub-probes comprise sets of three or more sensors, and the sensors in each set expand outward in different directions. The expansion mechanism is, for example, a basket mechanism, or any other expansion mechanism known to the art of intravascular probes. Optionally, the expansion mechanism includes any of the features described herein for the leaf springs with a pair of sensors, for example pulling on a single wire causes more than one sub-probe to expand.

The mechanism by which inner wire 114 and sheath 116 make the pairs of leaf springs expand is shown more clearly in FIGS. 1B and 1C, and in FIG. 2, which shows inner wire 114, sheath 116, and leaf springs 112 in a more detailed side cross-sectional view. Leaf springs 110 have a distal end 118 and a proximal end 120, while leaf springs 112 have a distal end 122 and a proximal end 124. Sheath 116 is joined to proximal ends 120 and 124. Inner wire 114 is joined to the distal ends 118 and 122. When inner wire 114 is pulled back (to the right in FIG. 2) relative to sheath 116, the distal end of each pair of leaf springs is pulled toward the proximal end, and each pair of leaf springs is forced to expand.

Optionally, pulling back on inner wire 114 relative to sheath 116 allows the center of each sub-probe to remain substantially in a fixed position in the blood vessel. This can be accomplished, for example, if sheath 116 is sufficiently rigid and resistant to buckling so that, when inner wire 114 is pulled and sheath 116 is pushed, the distance between proximal ends 120 and 124 remains constant. There is also optionally a mechanism, not shown in the drawings, near the proximal end of the catheter outside the body, which allows a doctor to make a single manipulation which pulls inner wire 114 back and simultaneously pushes sheath 116 forward by the same distance. This allows the mid-point between distal end 118 and proximal end 120, and the mid-point between distal end 122 and proximal end 124, to both remain fixed in place when the probe is expanded.

Inner wire 114 is exposed at distal end 118, which is the distal end of the whole probe, because sheath 116 ends before distal end 118, as may be seen in FIG. 1C. At distal end 122, there is an opening running along the bottom of sheath 116, through which inner wire 114 is exposed, so that inner wire 114 can be connected to distal end 122. Sheath 116 goes through a hole near distal end 122, so it can move freely back and forth without interference from distal end 122. Inner wire 114 moves freely back and forth without interference from proximal ends 120 and 124, because inner wire 114 is inside sheath 116, which passes through proximal ends 120 and 124.

Alternatively, instead of sheath 116 being coupled to the proximal ends, and inner wire 114 being coupled to the distal ends, sheath 116 is coupled to the distal ends, and inner wire 114 is coupled to the proximal ends, and is pushed, rather than pulled, to expand the probe. Alternatively, instead of a sheath and an inner wire, there are two wires side by side, one of them coupled to the distal ends of the leaf springs, and one of them coupled to the proximal ends. A potential advantage of having a sheath coupled to the proximal ends, and an inner wire coupled to the distal ends, is that the sheath, which pushes when the leaf springs are expanded and is therefore subject to buckling, has a higher buckling limit than the inner wire. A low buckling limit for the inner wire is generally not significant because the inner wire pulls when the leaf springs are expanded. In fact, the inner wire under tension helps to stabilize the sheath against buckling. It is noted that the sheath would not be as effective at stabilizing the inner wire against buckling, if the inner wire were pushing against the proximal ends and the sheath were pulling against the distal ends, because the inner wire could buckle through an opening in the sheath, in the regions where the inner wire is coupled to the leaf springs.

If the inner wire and sheath are both coupled rigidly to the leaf springs, then the degree of expansion of leaf springs 110 will have a fixed relationship to the degree of expansion of leaf springs 112. For example, if the two pairs of leaf springs have the same geometry, then one pair of leaf springs will always expand by the same amount as the other pair of leaf springs. The expansion would depend only on how far inner wire 114 is pulled relative to sheath 116. However, this may not be desirable, since the blood vessel may not have the same diameter everywhere along the length of the probe. For example, suppose the blood vessel is narrower at the location of sensors 102 and 104, than it is at the location of sensors 106 and 108. If one pair of leaf springs always expands by the same amount as the other pair, and if they do not exert enough force on the wall of the blood vessel to significantly deform it, then when sensors 102 and 104 reach the blood vessel wall they will stop, and sensors 106 and 108 will not be able to reach the blood vessel wall. Sensors 106 and 108 may not be able to obtain good images of the wall if they are too far away from it. If the leaf springs at the narrow portion exert enough force on the blood vessel wall to significantly distort the blood vessel, then it may be possible for sensors 102, 104, 106 and 108 to all touch the blood vessel wall, by deforming the blood vessel where it is narrow, next to sensors 102 and 104. However, this could be dangerous if there is fragile plaque in the walls of the blood vessel.

In order to allow each sub-probe to adapt to the diameter of the blood vessel at the location of that sub-probe, without distorting the blood vessel, one or both of inner wire 114 and sheath 116 are optionally not rigidly coupled to the leaf springs. Instead, at least one of them, for example inner wire 114, is coupled flexibly to the leaf springs, by means of coil springs for example. This is shown schematically in FIG. 2, which shows how inner wire 114 and sheath 116 are coupled to leaf springs 112.

In FIG. 2, sheath 116 is rigidly attached to proximal end 124 of leaf springs 112, and sheath 116 of the catheter pushes proximal end 124 to the left, toward distal end 122 of leaf springs 112. Optionally, a ridge 202 attached to and going around sheath 116 provides a surface for proximal end 124 to push against. A hole 204 near distal end 122 of leaf springs 112 allows sheath 116 to pass through distal end 122 without exerting any force on it.

A block 208 is attached to inner wire 114 to the left of distal end 122. When inner wire 114 is pulled to the right relative to sheath 116, block 208 pushes to the right against spring 210, which pushes against distal end 122.

If there were no spring 210, and neglecting any stretching, compression, or buckling of inner wire 114 and sheath 116, the amount that one pair of leaf springs is open would fix the amount that the other pair of leaf springs is open, even if the inner diameter of the blood vessel were different for the two pairs of leaf springs. Including spring 210 between block 208 and distal end 122 of leaf springs 112, and/or a similar spring at the distal end of leaf springs 110, makes it possible for the two pairs of leaf springs to open by different amounts, depending on the forces they encounter.

The more spring 210 is compressed, the greater force it will exert on distal end 122, and the greater force leaf springs 112 will exert on the wall of the blood vessel. The force that leaf springs 112 exert on the blood vessel wall depends on the difference in the inner diameter of the blood vessel at the axial positions of the sensors, and on the spring constant of spring 210.

Optionally, the spring constant of spring 210 is chosen to have a value so that the pair of leaf springs at the narrower diameter will push sensors 106 and 108 against the blood vessel wall with a great enough force to keep them firmly in place when they collect imaging data, but with a force that is not so great that the sensors will significantly deform the wall of the blood vessel. In particular the force is preferably not great enough to break any plaque, which could be dangerous. For example, the probe does not press on the wall with a pressure greater than 1 atmosphere, or 0.5 atmospheres, or 2 atmospheres. Thus, in addition to allowing the different leaf springs to open by different amounts, spring 210 also serves a safety function.

As shown in FIG. 2, spring 210 is under compression. Alternatively, spring 210 is under tension, for example by having block 208 and spring 210 to the right of distal end 122, between leaf springs 112, and attaching spring 210 to the back (right side) of distal end 122.

Optionally, there is a spring between ridge 204 and proximal end 124, instead of or in addition to spring 210 between block 208 and distal end 122. The springs need not be coil springs as shown in the drawing, but could be any kind of flexible coupling. The springs, if they are found only on one end (distal or proximal) of each leaf spring, need not be found on the same end of each leaf spring.

A hole 206 at the bottom of sheath 116, extending some distance to both sides of distal end 122, exposes inner wire 114 of the catheter. Hole 206 need not extend very far azimuthally around sheath 116, as it seems to do in FIG. 2. In fact, if hole 206 does not extend too far around sheath 116 azimuthally, then sheath 116 will be more resistant to buckling, which is potentially an advantage. Making hole 206 shorter will also make sheath 116 more resistant to buckling. However, if hole 206 is too narrow or too short, then sheath 116 may interfere with the coupling between inner wire 114 and distal end 122, for some range of expansion states of leaf springs 112.

Flexible coupling between the catheter and the expansion mechanism, as exemplified by spring 210, is also optionally used if a different expansion mechanism is used. For example, instead of a pair of leaf springs, the expansion mechanism could be a basket, or any other expansion mechanism known to the art.

Optionally, instead of or in addition to there being a flexible coupling such as spring 210 between the catheter and the leaf springs, the leaf springs themselves are sufficiently flexible so that different pairs of leaf springs can open by different amounts, and so that the force exerted by the sensors on the blood vessel wall is not too great.

Optionally, leaf springs 110, and 112 (or whatever expansion mechanisms are used) are made of a superelastic material, such as superelastic NiTi. Superelastic materials are shape-memory materials that are used not too far above their martensite to austenite transition temperature, so that they revert to martensite, and undergo a large strain, when a relatively small stress is applied to them. Alternatively, one or more of the leaf springs are made of a material that is not superelastic, such as 304 or 316 stainless steel, or other biocompatible materials such as alloys based on cobalt, titanium or tantalum which are used in stents. It should be noted, however, that stents are generally made of materials with a low yield stress, so that they remain in an expanded state when the expanding force is removed, while for the leaf springs it is preferable to use a material which does not exceed its yield stress in the course of expanding, so that they will contract again when the expanding force is removed. Hence, some materials that are useful for stents may not be useful for the leaf springs, and vice versa.

A potential advantage of using a superelastic material is that the leaf springs can undergo a large displacement, as much as several percent for NiTi, without undergoing plastic deformation. Non-superelastic alloys, by contrast, have yield strains typically less than 0.2%. Although in principle a leaf spring made of a non-superelastic material could also undergo a large displacement without plastic deformation, if the leaves of the leaf spring are thin enough, such thin leaves may be difficult to manufacture, and the leaf spring might not exert enough force on the blood vessel wall to hold the probe in place, unless the leaves are made of a material with very high elastic modulus. With superelastic materials, the leaves may be made thicker, and such high elastic modulus is not needed. Optionally, the probe shown in FIGS. 1A, 1B and 1C increases in diameter by a factor of at least 1.5 from its contracted state to its fully expanded state. Optionally, it increases in diameter by at least a factor of 2, or at least a factor of 3. For example, the probe has a diameter of 4 French (1.33 mm) or 5.5 French (1.83 mm), when contracted, and it has a diameter greater than 2.8 mm, or greater than 3.5 mm, or greater than 4.5 mm, or greater than 6 mm, when fully expanded. Using superelastic material for the leaf spring may be particularly advantageous when such large ratios of expanded diameter to contracted diameter are desired.

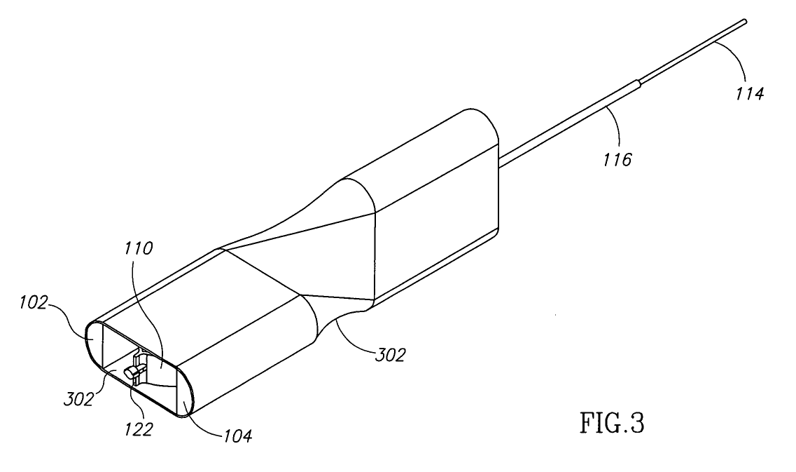

FIG. 3 shows the same probe shown in FIG. 1C, but covered by probe sheath 302 to keep non-biocompatible materials in the probe out of contact with the blood. Optionally, probe sheath 302 is made of a stretchable, biocompatible material, such as silicone of thickness for example between 10 and 100 micrometers, polyurethane, SCBS, or a composite material, to allow it to stretch to accommodate the expansion of the probe. Alternatively, the probe sheath is not very stretchable, but fits loosely around the probe when the probe is in its compressed state, and fits more tightly around the probe when the probe is expanded.

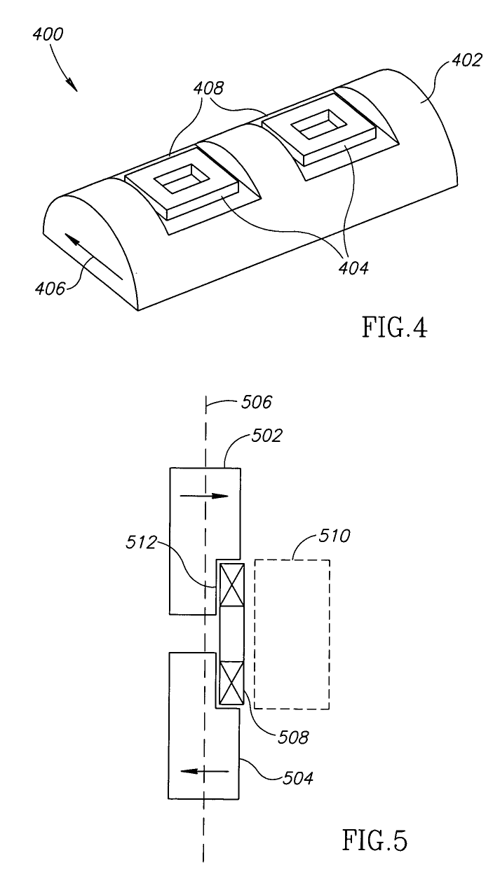

Note that the probe in FIG. 3 expands against the blood vessel walls to hold the probe in place and to bring sensors into proximity with the wall. However, the probe in FIG. 3 does not obstruct the flow of blood, but allows blood to flow around it, because (at any given point) it expands only along one axis. FIG. 4 shows an MRI sensor 400 which optionally is used as one of the imaging sensors in FIGS. 1A-1C. This sensor has the general shape of a semicircular cylinder, as in FIGS. 1A-1C. It is a self-contained MRI sensor, including a permanent magnet 402, and RF coils 404, which optionally are used both as transmitting and receiving coils. Alternatively, there are separate transmitting and receiving coils. Alternatively, one or more RF coil is replaced by a different kind of RF antenna. The direction of magnetization of the magnet is shown by arrow 406.

The coils are located in slots 408, which are cut out of the magnet. Alternatively, there is one long slot with a single long coil, instead of two separate slots each with its own coil. Having two slots each with its own coil has the potential advantage that, for the same longitudinal extent of the field of view of the sensor, less of the magnet volume is missing, especially near the imaging region close to the surface of the probe, so the magnet produces a greater magnetic field in the imaging region. Having one long slot with a single long coil has the potential advantage that, for the same ohmic heating, the RF field is greater.

Optionally there is more than one coil in each slot. For example, if there are separate transmitting and receiving coils, then optionally there is a transmitting coil and a receiving coil in the same slot. The part of the magnet that is removed to make the slots would contribute relatively little to the static magnetic field in the imaging region, which is just above the coils. Using this volume for the coils, rather than putting the coils outside a cylindrical magnet without slots as in the prior art, allows the magnet to be brought closer to the imaging region, more than making up for any loss in magnetic moment of the magnet due to the slots.

Allocating a larger volume for the coils enables stronger RF fields to be produced with a same amount of ohmic heating, or the same RF fields with less ohmic heating. Strong RF fields in short pulses (i.e. high bandwidth) are potentially advantageous, especially if the static magnetic field is very inhomogeneous, because they make it possible to excite a larger volume, and to obtain more spin echoes within a given time, producing a higher signal to noise ratio. Strong RF fields also make it possible to refocus the magnetic moments of the nuclei quickly, and, particularly with a high RF bandwidth, the nuclei do not diffuse away from the resonant region before they can be refocussed. Ohmic heating of the RF coils, which can affect the magnetization of permanent magnets as well causing heating of body tissue, may be the factor which limits how strong the RF fields are. In other cases, however, direct RF heating of body tissue is the limiting factor.

If the slot takes up too large a fraction of the magnet volume, however, then the static magnetic field will be weaker because the magnet volume will be smaller, for a given envelope of the probe, and a lower static magnetic field may result in lower signal to noise ratio. Optionally, the shape and size of the slot and coils are optimized, for a given probe envelope, in order to maximize some measure of probe performance, for example the signal to noise ratio that can be obtained in a given data acquisition time. Such optimization of the design may be done, for example, using software to simulate the probe performance and to calculate the RF and static magnetic field distribution.

Optionally, the magnet is made of a sintered material which has a relatively high resistivity, so the RF coils will not induce significant eddy currents in the magnet. Alternatively, the magnet is a good conductor, but using a magnet which is a good conductor has the potential disadvantage that eddy currents may partly cancel the RF magnetic field in the imaging region, and may heat the magnets to an undesirably high temperature.

The imaging region is above the probe, in the orientation of the probe shown in FIG. 4. The two RF coils are excited in phase, so the RF field is approximately vertical everywhere in the region above the probe, even between the coils. The probe optionally has any number of additional slots, all with RF coils excited in phase. The imaging region is not limited, azimuthally, to the region right above the center of the coils, where the RF field is vertical and the static magnetic field is horizontal (opposite in direction to the direction of magnetization of the magnet). Above and a little to the sides of the coils, the RF magnetic field has a horizontal component, but the static magnetic field has a vertical component, and the RF magnetic field is still nearly perpendicular to the static magnetic field, so that most of the RF field contributes to exciting the nuclei. The configuration shown in FIG. 4 thus makes efficient use of the static and RF magnetic fields.

The use of slots for RF coils in an MRI probe is not limited to the magnet configuration shown in FIG. 4. It can also be used for other magnet configurations, for example with the magnetization in the longitudinal direction, or having a component in the longitudinal direction, and with a plurality of magnets having different directions of magnetization. For example, FIG. 5 shows a side cross-sectional view of probe with two magnets 502 and 504, arranged longitudinally, with opposite directions of magnetization perpendicular to a longitudinal axis 506. An RF coil 508 produces an RF magnetic field which is approximately perpendicular to the static magnetic field produced by the magnets, in an imaging region 510. A slot 512 extends over parts of both magnets, and RF coil 508 is located in slot 512, rather than beyond the outermost surface of magnets 502 and 504. As with the probe shown in FIG. 4, slot 512 allows the magnets to be brought closer to a blood vessel wall, or to whatever is being imaged. Another example of an MRI probe with a slot is shown in FIG. 1A of a patent application titled “Magnetic Coil Configurations for MRI Probes,” attorney's docket number 334/03511, filed on even date, at the US Patent and Trademark Office.

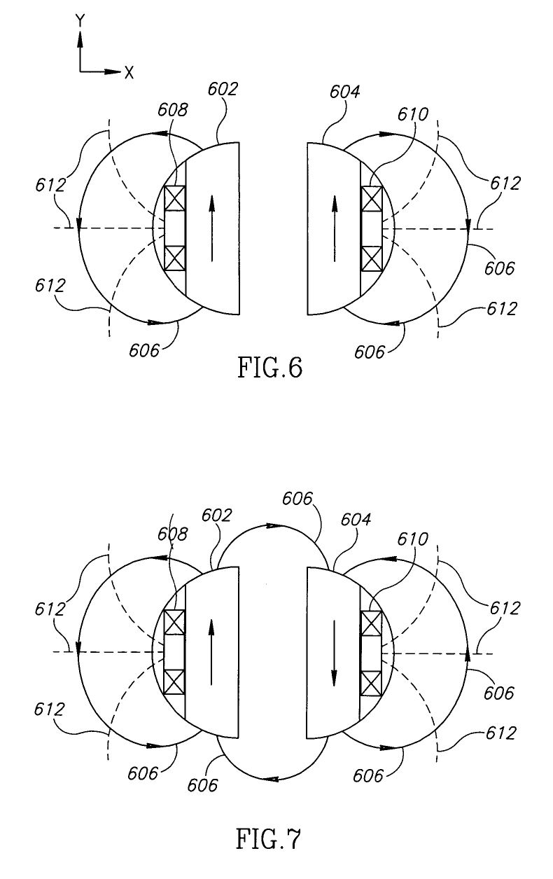

FIGS. 6 through 16 show cross-sectional views, each in a plane perpendicular to the longitudinal axis, of magnet and RF coil configurations for probes with MRI sensors, according to various exemplary embodiments of the invention. In FIG. 6 there are two magnets 602 and 604, each resembling the sensor shown in FIG. 4. The two magnets are magnetized in the same direction, the +y direction, and they move respectively in the +x and −x directions when the probe expands. Some of the field lines 606 of the static magnetic field are shown. Magnet 602 has an associated RF coil 608, and magnet 604 has an associated RF coil 610. Optionally, the RF coils are in slots in the magnets, as in FIG. 4, and optionally each RF coil shown in FIG. 6 represents two or more RF coils arranged longitudinally, as in FIG. 4. RF magnetic field lines 612 are shown as dashed lines in FIG. 6. The RF magnetic field lines are drawn assuming that the magnet is made of a sintered material which will not produce significant eddy currents. Although the exact form of the RF magnetic fields depends on the phase difference between the two coils, in practice the imaging region of each sensor is much closer to one RF coil than to the other, so qualitatively the RF fields will be of the shape shown, dominated by the nearest RF coil, and with minor changes depending on the phase difference between the two coils.

Note that, over a fairly broad imaging region near each of the magnets and coils, the RF magnetic field is approximately perpendicular to the static magnetic field. Only the component of RF field that is perpendicular to the static magnetic field contributes to excitation of nuclei in NMR, so the configuration shown in FIG. 6 makes efficient use of the static and RF magnetic fields. The configuration would be much less efficient if the magnets were magnetized in the +x or −x direction.

Magnets 602 and 604 repel each other, which is an advantage when the magnet is expanding, because, assuming the probe uses the expansion mechanism shown in FIGS. 1A-1C and 2, it is not necessary to pull on the inner wire of the catheter with so much force. This is especially true initially, when the magnets are close together, and when static friction between the inner wire and sheath of the catheter may significantly increase the force needed to start making the probe expand. Although the repulsion between the magnets means that more force is needed to compress the probe again, that force is supplied by the spring force of the leaf springs when the physician reduces the force pulling on the inner wire of the catheter. For safety reasons, the probe is in its collapsed state when there is no force pulling on the inner wire of the catheter. Another potential advantage of having the magnets repel each other is that the force pulling on the inner wire is a monotonic function of the degree of expansion of the probe, so it may be easier to make the probe expand in a controlled way. Alternatively, the magnets in FIG. 6 are oriented so that they attract each other, as shown in FIG. 7.

The configuration in FIG. 6 can be generalized to more than two magnets. For example, FIG. 8 has four magnets 902, 904, 906 and 908, with every pair of adjacent magnets repelling each other. RF coils 910, 912, 914 and 916 are located on the outside of the probe, one RF coil for each magnet. As in FIG. 6, the RF magnet field lines 612 are nearly perpendicular to the static magnetic field lines 606, so the configuration in FIG. 8 makes efficient use of the static and RF magnetic fields. However, the configuration shown in FIG. 6, with only two magnets, has the potential advantage that the probe may not occlude the blood vessel as much when the probe is expanded. This may be especially true if the probe is covered with a sheath, as in FIG. 3.

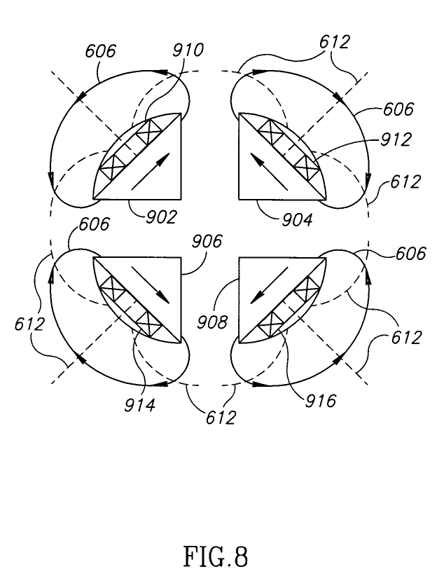

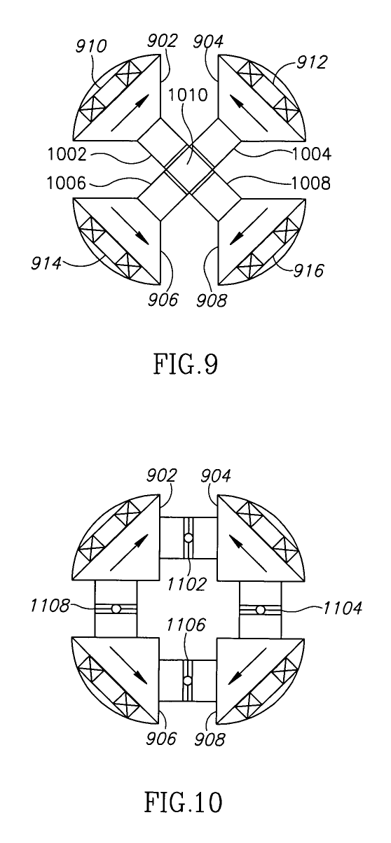

FIGS. 9 and 10 show expansion mechanisms that are used in two different embodiments of the invention, both using the magnet and coil configuration of FIG. 8. In FIG. 9, each of the four magnets is mounted on one leaf spring, and the four leaf springs 1002, 1004, 1006, and 1008 join together at each end. Optionally, as shown in FIG. 9, there is a small cube or parallelopiped 1010, and one end of each leaf spring is attached to one side of the cube. There is another cube at the other end (not visible in FIG. 9), to which the other ends of the leaf springs are attached. As in FIG. 2, there is optionally a catheter with an inner wire and outer sheath, attached respectively to the cube at the distal end and the cube at the proximal end, and pulling on the inner wire makes the probe expand. Alternatively, a ring is used instead of a cube. Alternatively, any other mechanical expanding structure known in the art is used for the expansion mechanism, for example any of the expanding cylindrical structures used for stents, and the four magnets are mounted on the outside.

Two other examples of a probe with four MRI sensors, with an expanding structure, but with the sensors all at different longitudinal locations, are shown in FIGS. 3 and 4 of a patent application titled “Magnetic Coil Configurations for MRI Probes,” attorney's docket number 334/03511, filed on even date, at the US Patent and Trademark Office.

In FIG. 10 there are four pairs of leaf springs, 1102, 1104, 1106 and 1108, each pair resembling the pair of leaf springs 110 in FIGS. 1B and 1C, for example. Each pair of leaf springs is mounted between two adjacent magnets. Optionally, there is a catheter with an inner wire and a sheath, as in FIGS. 1A-1C and FIG. 2. Optionally, the inner wire and the sheath each divide into four parts, and the four parts of the inner wire are attached respectively to the distal ends of the four pairs of leaf springs, while the four parts of the sheath are attached respectively to the proximal ends of the four pairs of leaf springs. Pulling on the inner wire thus causes all four pairs of leaf springs to expand, pushing all four magnets away from each other. Optionally, all the pairs of leaf springs have the same design, so that when the inner wire is pulled, all four pairs of leaf springs expand by the same amount, and the probe expands symmetrically.

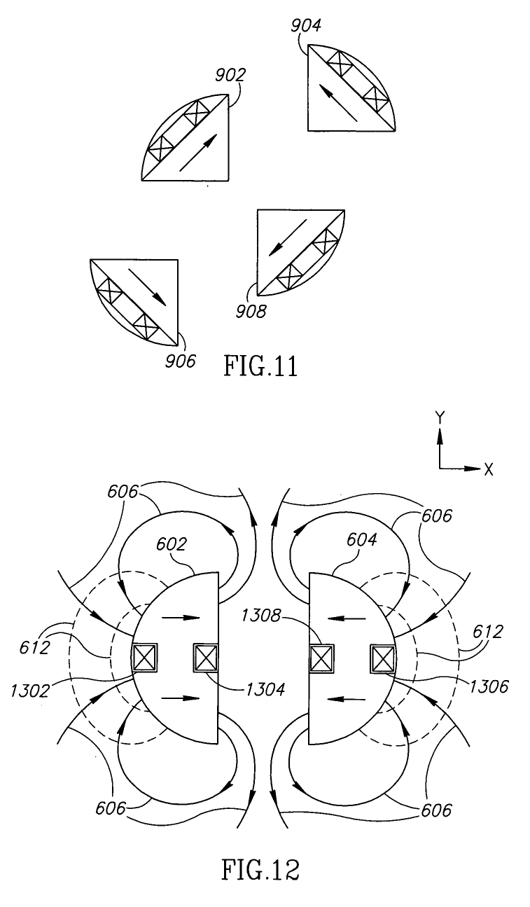

A potential disadvantage of the configuration in FIG. 8 is that, although adjacent magnets repel each other, diagonally opposite magnets attract each other. Consequently, the probe may be subject to a mechanical instability in which the magnets move into a configuration as shown in FIG. 11, in which two diagonally opposite magnets move close together and the other two magnets move away. Optionally, the expansion mechanism, whether it resembles that shown in FIG. 9 or that shown in FIG. 11, is stiff enough to stabilize the probe against the mode shown in FIG. 11.

FIG. 12 shows a probe with two magnets 602 and 604, magnetized in the +x and −x directions, but with a different RF coil configuration than FIG. 6, which makes the RF magnetic field approximately perpendicular to the static magnetic field over a broad imaging region. Magnet 602 has a slot 1302 on the outer surface and a slot 1304 on the inner surface, both slots running the length of the magnet. A coil is located in the two slots, with current running up one slot, for example, over the top of the magnet (not shown in FIG. 12), down the other slot, and around the bottom of the magnet to the first slot. (In this description, “up” and “down” refer to directions perpendicular to the plane of the drawing, and the “top” and “bottom” of the magnet mean the two ends of the magnet, for example the distal end and the proximal end.) Magnet 604 has similar slots 1306 and 1308, with a coil in them. The RF magnetic field lines 612 are approximately perpendicular to the static magnetic field lines 606, which means that the configuration makes efficient use of the fields.

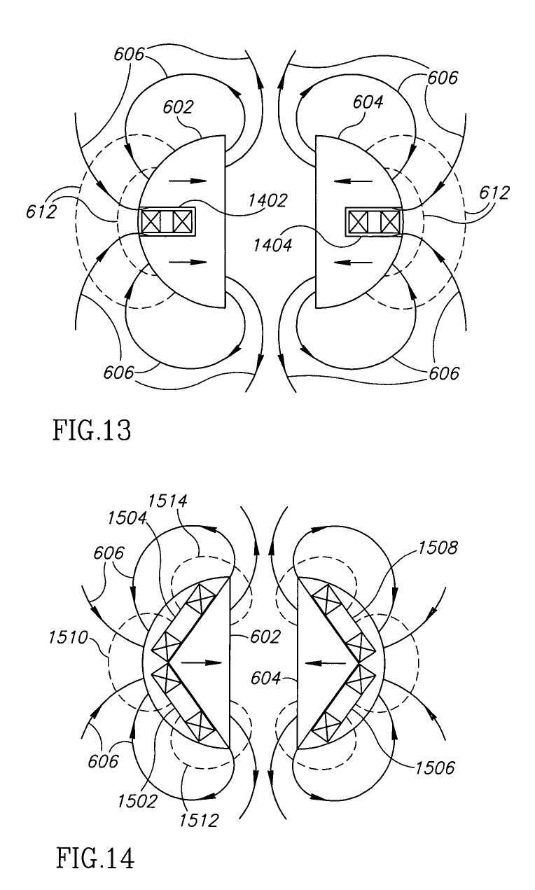

FIG. 13 shows a probe similar to FIG. 12, but each magnet has only a single slot on its outer side, slot 1402 for magnet 602 and slot 1404 for magnet 604. Each slot has an entire coil, with current running up one side and down the other side, so there is no need for the coil to extend around the ends of the magnets, as occurs in the configuration of FIG. 12. The RF field lines 612 and static magnetic field lines 606 are similar to those in FIG. 12.

In FIG. 14, each magnet has two RF coils side by side on the outer side of the magnet, optionally in a slot going across the magnet, similar to the slots shown in FIG. 4. Magnet 602 has RF coils 1502 and 1504, while magnet 604 has RF coils 1506 and 1508. The magnets are magnetized in the +x and −x directions, as in FIGS. 12 and 13. For each magnet, the two pairs of RF coils are excited 180 degrees out of phase. Thus, adjacent to magnet 602, RF field line 1510 links coils 1502 and 1504, while other RF field lines 1512 and 1514 each link only one of the coils. The static magnetic field lines 606 are similar to those shown in FIGS. 12 and 13. Over a broad region around the outside of each magnet, the RF magnetic field lines are approximately perpendicular to static magnetic field lines 606, meaning that the probe makes efficient use of the fields.

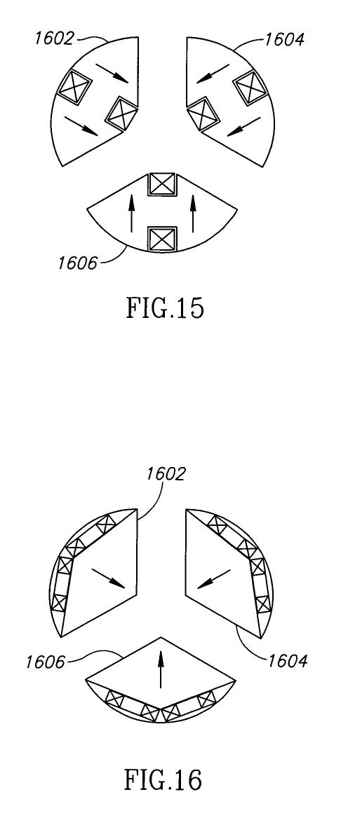

The configurations shown in FIGS. 12, 13 and 14 can be generalized to configurations involving an odd or even number of magnets. In these configurations, the magnets are all magnetized radially inward, or all magnetized radially outward, and every magnet repels every other magnet, whether adjacent or not. FIG. 15, for example, has three magnets 1602, 1604 and 1606, each magnet having the shape of a 120 degree sector of a circular cylinder, and all magnets being magnetized radially inward. The RF coil configuration in FIG. 15 is similar to that in FIG. 12, with two slots in each magnet, and an RF coil running through the two slots. Alternatively, there is only a single slot in each magnet, with a coil in it, as in FIG. 13. For reasons of clarity, the field lines of the static and RF magnetic fields are not shown in FIG. 15, but they look similar to the field lines shown in FIG. 12, only repeating every 120 degrees instead of every 180 degrees.

FIG. 16 shows a similar configuration with three magnets, but with the RF coils resembling the configuration in FIG. 14. There are two coils, side by side, on the outside of each magnet. The static and RF magnetic fields lines, not shown in FIG. 16, would be similar to those shown in FIG. 14, but repeating every 120 degrees instead of every 180 degrees. In the configurations shown in FIGS. 15 and 16, as in FIGS. 12, 13 and 14, the RF magnetic fields are approximately perpendicular to the static magnetic fields throughout a broad imaging region outside each magnet. The configurations shown in FIGS. 15 and 16 with three magnets can be generalized to configurations involving four or more magnets, as will be understood by one skilled in the art.

Optionally, any of the probes described here have radio-opaque markings, which are used to precisely locate the probe, for example with a fluoroscope, and thus to correlate the images made by the probe with the position of the probe in the body.

In describing the geometry of probes, sensors, or other bodies, “substantially” as used herein, in particular in the claims, means “to within 10% of the diameter” of the probe, sensor, or other body being described. As used herein, “cylinder” and cylindrical” do not necessarily refer to a right circular cylinder, unless explicitly stated as such.

In describing probes covered with a sheath, the sheath is considered part of the probe. Hence, describing a probe as touching a wall does not necessarily mean that a part of the probe such as an imaging sensor touches the wall directly, but it also includes the case where only the sheath touches the wall directly.

Describing the at least one magnets of an MRI sensor as “substantially comprising only a single magnet, uniformly magnetized in a single direction,” means that any lack of uniformity in the direction or magnitude of magnetization of the at least one magnets does not change the magnetic field in the imaging region enough to substantially affect the operation of the sensor, and any discontinuity in the magnets, for example if the magnets comprise two magnets touching each other or separated by a thin layer of glue, does not cause the operation of the probe to differ substantially from what it would be if the magnets comprised a single continuous magnet.

The invention has been described in the context of the best mode for carrying it out. It should be understood that not all features shown in the drawings or described in the associated text may be present in an actual device, in accordance with some embodiments of the invention. Furthermore, variations on the method and apparatus shown are included within the scope of the invention, which is limited only by the claims. Also, features of one embodiment may be provided in conjunction with features of a different embodiment of the invention. As used herein, the terms “have”, “include” and “comprise” or their conjugates mean “including but not limited to.”

1. An imaging probe for imaging inside a cavity surrounded by a wall, the probe comprising:
a) a probe body having a contracted state and an expanded state; and
b) at least two imaging sensors, mounted on the probe body and having fields of view in different directions;
wherein, when the probe body is in the expanded state, the fields of view of the imaging sensors respectively comprise portions of the wail of the cavity on different sides of the cavity.

2. An imaging probe according to claim 1, wherein, when the probe body is in the expanded state, the at least two imaging sensors are displaced from each other toward the wall, from their position when the probe body is in the contracted state.

3. An imaging probe according to claim 1, adapted for inserting into a blood vessel and using the blood vessel as the cavity.

4. An imaging probe according to claim 3, including a biocompatible sheath which covers the probe.

5. An imaging probe according to claim 4, wherein the sheath keeps blood from coming into contact with and flowing through the probe body.

6. An imaging probe according to claim 5, wherein when the probe body is in the expanded state, the probe touches the wall of the blood vessel in two contact regions on two opposite sides of the vessel, while leaving at least one free region, where the probe is not in contact with the wall, between the contact regions, thereby allowing blood to flow around the probe through the at least one free region.

7. An imaging probe according to claim 1, wherein the probe has a diameter between 1 and 2 mm in its contracted state.

8. An imaging probe according to claim 1, wherein the probe has a diameter between 2 mm and 6 mm in its expanded state.

9. An imaging probe according to claim 1, wherein the diameter of the probe in its expanded state is at least 1.5 times the diameter of the probe in its contracted state.

10. An imaging probe according to claim 1, wherein the imaging sensors are MU sensors, each sensor comprising:
a) at least one static magnetic field source which creates a static magnetic field in the field of view of said sensor, and
b) at least one RF coupling element, wherein at least one of the at least one RF coupling elements is capable of creating a time-varying magnetic field which is capable of exciting nuclei in the field of view of said sensor, and at least one of the at least one RF coupling elements is capable of receiving NMR signals from said excited nuclei and generating NMR electrical signals therefrom.

11. An imaging probe according to claim 10, wherein at least one of the at least one RF coupling elements comprises an antenna.

12. An imaging probe according to claim 11, wherein the antenna comprises a coil.

13. An imaging probe according to claim 10, wherein at least one of the at least one RF coupling elements uses the Hall effect.

14. An imaging probe according to claim 10, wherein at least one of the at least one RF coupling elements uses the magneto-optical effect.

15. An imaging probe according to claim 10, wherein, for at least one of the MRI sensors:
a) the at least one static magnetic field source comprises at least one permanent magnet;
b) the convex magnet volume, defined as a smallest convex volume which includes all of the at least one magnet, is cylindrical;
c) the at least one magnet substantially reaches all of the radial surface of the convex magnet volume, except for at least one slot, each slot being less than the length of the convex magnet volume; and
d) one of the at least one RF coupling elements is located in one of the at least one slots, substantially entirely within the convex magnet volume.

16. An imaging probe according to claim 10, wherein for each of the MU sensors, the at least one static magnetic field source comprises a permanent magnet.

17. An imaging probe according to claim 16, wherein the magnets of the two MRI sensors repel each other.

18. An imaging probe according to claim 17, wherein the magnets of the two MRI sensors are both magnetized in directions that are more than 45 degrees away from an axis along which the sensors move apart from each other when the probe body expands, and the magnets are magnetized in directions less than 90 degrees away from each other.

19. An imaging probe according to claim 17, wherein the magnets of the two MRI sensors are both magnetized in directions that are less than 45 degrees away from an axis along which the sensors move apart from each other when the probe body expands, and the magnets are magnetized in directions more than 90 degrees away from each other.

20. An imaging probe according to claim 10, wherein the time-varying magnetic field created by the at least one RF coupling element of each MRI sensor is oriented at an angle between 45 and 135 degrees from the direction of the static magnetic field created by the static magnetic field source of said MRI sensor, at at least one location in the field of view of said MRI sensor.

21. An imaging probe according to claim 1, wherein the imaging sensors are ultrasound imaging sensors.

22. An imaging probe according to claim 1, wherein the at least two imaging sensors comprise exactly two imaging sensors.

23. An imaging probe according to claim 1, wherein the at least two imaging sensors comprise at least three imaging sensors.

24. An imaging probe according to claim 23, wherein the probe body comprises a plurality of expansion mechanisms, each expansion mechanism attached to at least two but not all of the imaging sensors, such that when each expansion mechanism causes the imaging sensors to which it is attached to move apart from each other, the probe body expands.

25. An imaging probe according to claim 24, wherein the imaging sensors are arranged in a circle, and one of the expansion mechanisms is located between, and attached to, each pair of adjacent imaging sensors in the circle.

26. An imaging probe according to claim 25, wherein at least one expansion mechanism comprises a pair of leaf springs.

27. An imaging probe according to claim 24, wherein at least one of the expansion mechanisms comprises shape memory alloy.

28. An imaging probe according to claim 23, wherein the probe body comprises a single centrally located expansion mechanism which is attached to all the sensors, and causes the sensors to move apart from each other, expanding the probe.

29. An imaging probe according to claim 28, wherein the expansion mechanism comprises a basket comprising a plurality of arms, each arm attached to exactly one sensor and each sensor attached to exactly one arm.

30. An imaging probe according to claim 28, wherein the expansion mechanism comprises shape memory alloy.

31. An imaging probe according to claim 30, wherein raising the temperature of the shape memory alloy above its transition temperature causes said expansion mechanism to expand.

32. An imaging probe according to claim 30, wherein said expansion mechanism operates using a superelastic effect of the shape memory alloy.

33. An imaging probe according to claim 1, wherein the probe body comprises an expansion mechanism which causes the two sensors to move apart from each other, expanding the probe.

34. An imaging probe according to claim 33, wherein the expansion mechanism comprises a pair of leaf springs joined at both their ends and free in their middle portions, and each sensor is attached to the middle portion of a different one of the leaf springs, and not attached to the other leaf spring.

35. An imaging system comprising an imaging probe according to claim 1, and a catheter adapted for inserting the imaging probe into the cavity.

36. An imaging system according to claim 35, wherein the catheter comprises a control cable, and manipulating the control cable causes the probe body to expand and contract.

37. An imaging system comprising a plurality of sub-probes, each sub-probe being an imaging probe according to claim 1, and a catheter adapted for inserting the sub-probes into the cavity.

38. An imaging system according to claim 37, wherein the catheter comprises a control cable, and manipulating the control cable causes the probe body of at least two of the sub-probes to expand and contract.

39. An imaging system according to claim 38, wherein the control cable is coupled to the sub-probes in a manner such that manipulating the control cable causes the probe bodies of a plurality of the sub-probes to expand simultaneously, and to contract simultaneously.

40. An imaging system according to claim 39, wherein, for each sub-probe in said plurality, one or both of said sub-probe and its coupling to the control cable is sufficiently flexible so that, when the control cable is manipulated, each sub-probe in said plurality expands to an extent that depends on the distance to the walls of the cavity, at the location of that sub-probe.

41. An imaging system according to claim 40, wherein for each sub-probe in said plurality, one or both of said sub-probe and its coupling to the control cable is sufficiently flexible so that, if the cavity is any artery the inner diameter of which varies between 2 mm and 4 mm at the locations of the sub-probes in said plurality, then all of the sub-probes in said plurality will touch the inner walls of the artery when the control cable is manipulated to cause said plurality of sub-probes to expand, without exerting a pressure of more than 1 atmosphere on the wall of the artery.

42. A method of producing images of the walls of a cavity, comprising:
a) introducing an imaging probe comprising a plurality of imaging sensors into the cavity;
b) causing the imaging probe to expand, causing the imaging sensors to move away from each other toward the walls;
c) generating imaging data by each imaging sensor in a different field of view, adjacent to that imaging sensor, of the walls of the cavity; and
d) reconstructing an image of the walls of the cavity from the imaging data.

43. A method according to claim 42, wherein introducing an imaging probe into the cavity comprises introducing the imaging probe into a lumen.

44. A method according to claim 43, wherein introducing the imaging probe into a lumen comprises introducing the imaging probe into a blood vessel.

45. A method according to claim 44, wherein causing the imaging probe to expand comprises causing the imaging probe to touch the wall of the blood vessels at a contact region, and leaving a free region where the imaging probe does not touch the blood vessel wall, allowing blood to flow around the imaging probe.

46. A method according to claim 43, wherein causing the imaging probe to expand comprises causing each of a plurality of sub-probes to expand by different amounts, depending on the inner diameter of the lumen at the location of each of said sub-probes.

47. A method according to claim 42, wherein introducing the imaging probe into the cavity comprises using a catheter.

48. A method according to claim 47, wherein causing the imaging probe to expand comprises manipulating the catheter.

49. A method according to claim 47, wherein generating imaging data comprises transmitting electrical power to the imaging probe through the catheter.

50. A method according to claim 47, wherein generating imaging data comprises receiving imaging data from the imaging probe through the catheter.

51. A method according to claim 42, wherein reconstructing an image comprises analyzing data by a data analyzer, and including transmitting the imaging data from the imaging sensors to the data analyzer, wherein the data from at least two of the sensors is transmitted on a same cable.

52. A method according to claim 51, wherein the sensing data from said two sensors is transmitted at different times.

53. A method according to claim 51, wherein the sensing data from said two sensors is transmitted in different frequency bands.

54. A method according to claim 51, including digitally encoding the data from said two sensors into different digital channels before transmitting it, and decoding the data from said two sensors after transmitting it, before analyzing it.

55.-137. (canceled)
